(19) United States
(12) Patent Application Publication
(10) Pub. No.: US 2025/0205211 A1
(43) Pub. Date: Jun. 26, 2025

(54) TYK2 INHIBITORS AND USES THEREOF

(71) Applicant: Alumis Inc., South San Francisco, CA (US)

(72) Inventor: Philip Nunn, South San Francisco, CA (US)

(21) Appl. No.: 18/852,039

(22) PCT Filed: Mar. 29, 2023
(86) PCT No.: PCT/US2023/016677

§ 371 Date: Sep. 27, 2024

Related U.S. Application Data
(60) Provisional application No. 63/324,754, filed on Mar. 29, 2022.
(60) Provisional application No. 63/344,126, filed on May 20, 2022.
(60) Provisional application No. 63/408,572, filed on Sep. 21, 2022.
(60) Provisional application No. 63/429,720, filed on Dec. 2, 2022.

(51) Int. Cl. A61K 31/4439; A61K 9/00; A61K 9/20; A61P 17/06; A61P 37/06

(52) U.S. Cl. CPC A61K 31/4439; A61K 9/0053; A61K 9/20; A61P 17/06; A61P 37/06

(57) ABSTRACT

Described herein are methods and compositions for treating a TYK2-mediated disorder. In some embodiments, the TYK2-mediated disorder is an autoimmune disorder, an inflammatory disorder, a proliferative disorder, an endocrine disorder, a neu-rological disorder, or a disorder associated with transplantation.

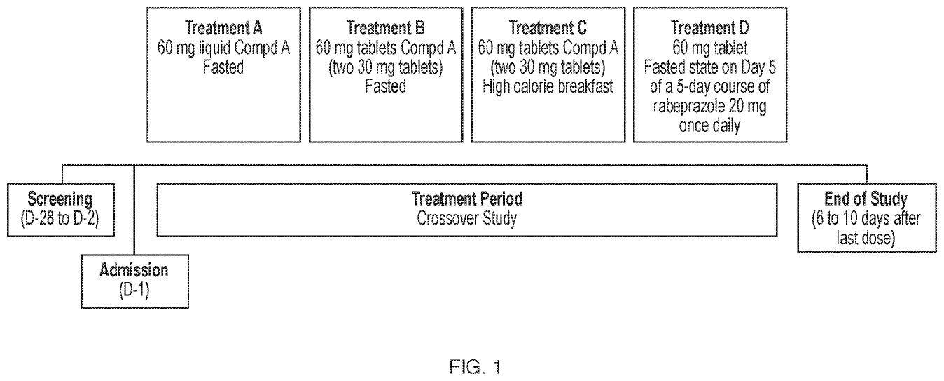

### CROSS REFERENCE TO RELATED APPLICATIONS

This application claims the benefit of, and priority to, U.S. Provisional Patent Application No. 63/324,754, filed on Mar. 29, 2022; U.S. Provisional Patent Application No. 63/344,126, filed on May 20, 2022; U.S. Provisional Patent Application No. 63/408,572, filed on Sep. 21, 2022; and U.S. Provisional Patent Application No. 63/429,720, filed on Dec. 2, 2022; the contents of each of which are hereby incorporated by reference herein in their entirety.

### BACKGROUND

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g., IFN-α, IFN-β, IFN-κ, IFN-δ, IFN-ε, IFN-τ, IFN-ω, and IFN-ζ ((also known as limitin), and interleukins (e.g., IL-4, IL-6, IL-10, IL-11, IL-12, IL-13, L-22, IL-23, IL-27, IL-31, oncostatin M, ciliary neurotrophic factor, cardiotrophin 1, cardiotrophin-like cytokine, and LIF). The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. A genome-wide association study of 2,622 individuals with psoriasis identified associations between disease susceptibility and TYK2. Knockout or tyrphostin inhibition of TYK2 significantly reduces both IL-23 and IL-22-induced dermatitis.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6.

Decreased TYK2 activity leads to protection of joints from collagen antibody-induced arthritis, a model of human rheumatoid arthritis. Mechanistically, decreased Tyk2 activity reduced the production of Th1/Th17-related cytokines and matrix metalloproteases, and other key markers of inflammation.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. This corroborates earlier studies linking increased TYK2 expression with MS susceptibility. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Genome-wide association studies of individuals with SLE versus an unaffected cohort showed highly significant correlation between the TYK2 locus and SLE.

TYK2 has been shown to play an important role in maintaining tumor surveillance and TYK2 knockout mice showed compromised cytotoxic T cell response, and accelerated tumor development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects.

Studies in T-cell acute lymphoblastic leukemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform.

Thus, selective inhibition of TYK2 has been suggested as a suitable target for patients with IL-10 and/or BCL2-addicted tumors, such as 70% of adult T-cell leukemia cases. TYK2 mediated STAT3 signaling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in postmortem brains of Alzheimer's patients. Inhibition of JAK-STAT signaling pathways is also implicated in hair growth, and the reversal of the hair loss associated with alopecia areata.

Accordingly, there is a need to provide methods for inhibiting the activity of TYK2 to treat one or more of the conditions described herein, without the side-effects associated with the inhibition of JAK2.

### SUMMARY

Described herein, in part, are methods and compositions that are useful in treating a TYK2-mediated disorder. For example, disclosed herein is a method of treating a TYK2-mediated disease or disorder in a patient in need thereof, comprising orally administering to the patient, once or twice daily, 30 mg, 40 mg, 60 mg, or 80 mg of a compound represented by:

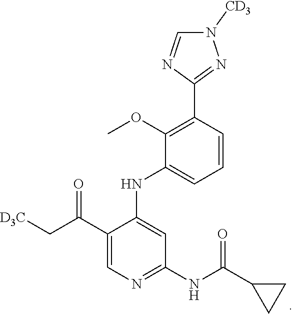 [SMILES: [2H]C([2H])([2H])CC(=O)c1cnc(NC(=O)C2CC2)cc1Nc1cccc(-c2ncn(C([2H])([2H])[2H])n2)c1OC]

Also disclosed herein is a method of treating one or more of: Crohn's disease, rheumatoid arthritis, psoriasis, systemic lupus erythematosus, ulcerative colitis, psoriatic arthritis, and systemic sclerosis in a patient in need thereof, comprising:
administering to the patient, once daily, two oral tablet compositions each comprising:
   30 mg of a compound represented by:

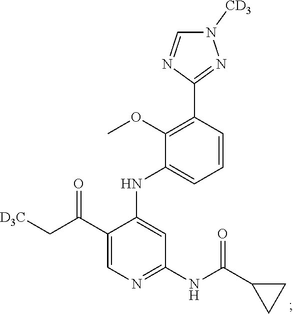 [SMILES: [2H]C([2H])([2H])CC(=O)c1cnc(NC(=O)C2CC2)cc1Nc1cccc(-c2ncn(C([2H])([2H])[2H])n2)c1OC]

and
a pharmaceutically acceptable excipient;
optionally orally administering to the patient rabeprazole; and
optionally administering food to the patient upon administration of the compound.

Further disclosed herein is a method of treating one or more of: Crohn's disease, rheumatoid arthritis, psoriasis, systemic lupus erythematosus, ulcerative colitis, psoriatic arthritis, and systemic sclerosis in a patient in need thereof, comprising:
orally administering to the patient, once daily or twice daily, 30 mg, 60 mg, or 80 mg of a compound represented by:

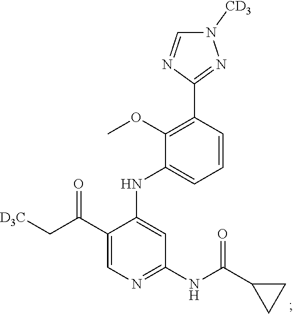 [SMILES: [2H]C([2H])([2H])CC(=O)c1cnc(NC(=O)C2CC2)cc1Nc1cccc(-c2ncn(C([2H])([2H])[2H])n2)c1OC]

wherein the patient is in a fasted state or a fed state.

Also disclosed here is fixed unit dose pharmaceutical composition for oral administration comprising:
30 mg, 40 mg, 60 mg, or 80 mg of a compound represented by:

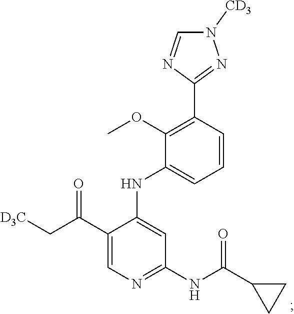 [SMILES: [2H]C([2H])([2H])CC(=O)c1cnc(NC(=O)C2CC2)cc1Nc1cccc(-c2ncn(C([2H])([2H])[2H])n2)c1OC]

and
a pharmaceutically acceptable excipient.

For example, disclosed herein is an oral tablet composition comprising:
about 30 mg of a compound represented by:

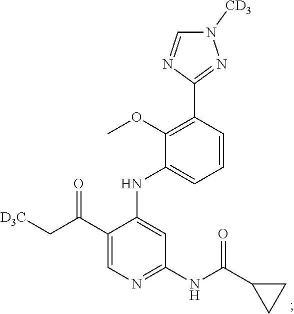 [SMILES: [2H]C([2H])([2H])CC(=O)c1cnc(NC(=O)C2CC2)cc1Nc1cccc(-c2ncn(C([2H])([2H])[2H])n2)c1OC]

and
a pharmaceutically acceptable excipient.

### BRIEF DESCRIPTION OF THE DRAWING

The FIGURE depicts a study investigating the relative bioavailability of Compound A as an oral tablet composition versus liquid formulation, and the effect of food or gastric acid reduction on the pharmacokinetics of a disclosed compound or oral tablet composition, in healthy participants.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein and in the appended claims, the singular forms “a,” “an,” and “the” include plural referents unless the context clearly dictates otherwise. Thus, for example, reference to “an agent” includes a plurality of such agents, and equivalents thereof known to those skilled in the art, and so forth. When ranges are used herein, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound or composition (e.g., an oral tablet composition) disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

### Methods and Compositions

Disclosed herein, for example, is a method of treating a TYK2-mediated disease or disorder in a patient in need thereof, comprising orally administering to the patient, once or twice daily, 30 mg, 40 mg, 60 mg, or 80 mg of a compound represented by:

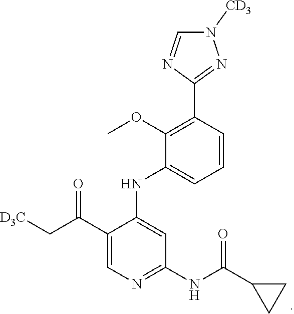 [SMILES: [2H]C([2H])([2H])CC(=O)c1cnc(NC(=O)C2CC2)cc1Nc1cccc(-c2ncn(C([2H])([2H])[2H])n2)c1OC]

In some embodiments, the methods described herein further comprise orally administering to the patient a proton pump inhibitor. For example, in some embodiments the proton pump inhibitor is selected from the group consisting of, e.g., rabeprazole, omeprazole, esomeprazole, lansoprazole, pantoprazole, and dexlansoprazole. In certain embodiments, the proton pump inhibitor is rabeprazole.

In some embodiments, a disclosed compound is administered to the patient in a fasted state. In other embodiments, the methods described herein further comprise administering food to the patient before administration or upon administration of a disclosed compound.

In further embodiments, the TYK2-mediated disease or disorder is selected from the group consisting of Crohn's disease, rheumatoid arthritis, psoriasis, systemic lupus erythematosus, ulcerative colitis, psoriatic arthritis, and systemic sclerosis.

For example, described herein is a method of treating one or more of: Crohn's disease, rheumatoid arthritis, psoriasis, systemic lupus erythematosus, ulcerative colitis, psoriatic arthritis, and systemic sclerosis in a patient in need thereof, comprising:
administering to the patient, once daily, two oral tablet compositions each comprising:
   30 mg of a compound represented by:

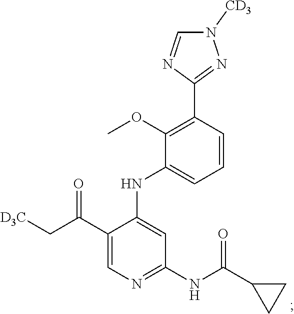 [SMILES: [2H]C([2H])([2H])CC(=O)c1cnc(NC(=O)C2CC2)cc1Nc1cccc(-c2ncn(C([2H])([2H])[2H])n2)c1OC]

and
a pharmaceutically acceptable excipient;
optionally orally administering to the patient rabeprazole; and
optionally administering food to the patient upon administration of the compound.

Also described herein is a method of treating one or more of: Crohn's disease, rheumatoid arthritis, psoriasis, systemic lupus erythematosus, ulcerative colitis, psoriatic arthritis, and systemic sclerosis in a patient in need thereof, comprising:
orally administering to the patient, once daily or twice daily, 30 mg, 60 mg, or 80 mg of a compound represented by:

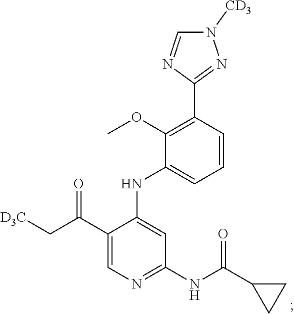 [SMILES: [2H]C([2H])([2H])CC(=O)c1cnc(NC(=O)C2CC2)cc1Nc1cccc(-c2ncn(C([2H])([2H])[2H])n2)c1OC]

wherein the patient is in a fasted state or a fed state.

In addition, disclosed herein is a fixed unit dose pharmaceutical composition for oral administration comprising:
30 mg, 40 mg, 60 mg, or 80 mg of a compound represented by:

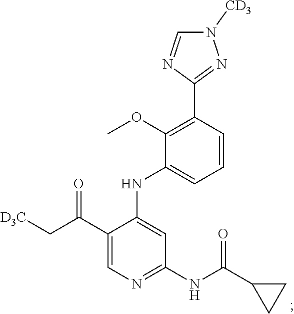 [SMILES: [2H]C([2H])([2H])CC(=O)c1cnc(NC(=O)C2CC2)cc1Nc1cccc(-c2ncn(C([2H])([2H])[2H])n2)c1OC]

and
a pharmaceutically acceptable excipient.

In some embodiments, a disclosed fixed dose pharmaceutical composition is formulated as a tablet.

Further disclosed herein is an oral tablet composition comprising:
about 30 mg of a compound represented by:

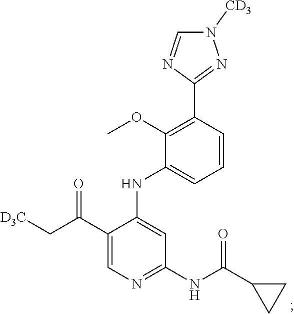 [SMILES: [2H]C([2H])([2H])CC(=O)c1cnc(NC(=O)C2CC2)cc1Nc1cccc(-c2ncn(C([2H])([2H])[2H])n2)c1OC]

and
a pharmaceutically acceptable excipient.

Also described herein are methods of treating a TYK2-mediated disease or disorder in a patient in need thereof, comprising administering to the patient an effective amount of a disclosed oral tablet composition. In some embodiments, the administrating is once daily administering. In other embodiments, the administrating is twice daily administering.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration (e.g., oral administration) and standard pharmaceutical practice as described, for example, in Remington: The Science and Practice of Pharmacy (Gennaro, 21^{st} Ed. Mack Pub. Co., Easton, PA (2005)).

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

For example, in some embodiments a disclosed compound or composition (e.g., an oral tablet composition) may be administered once daily to the patient. In other embodiments, a disclosed compound or composition (e.g., an oral tablet composition) may be administered twice daily to the patient.

In some embodiments, a disclosed compound or pharmaceutical composition is formulated for oral, topical (including buccal and sublingual), rectal, vaginal, transdermal, parenteral, intrapulmonary, intradermal, intrathecal, and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

In some embodiments, for example, a disclosed compound or pharmaceutical composition is formulated for oral administration. In some embodiments, the pharmaceutical composition is formulated as a tablet, a pill, a capsule, a liquid, a suspension, a dispersion, a solution, or an emulsion. In some embodiments, the pharmaceutical composition is formulated as a tablet, for example, a tablet formulated for oral administration.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involve the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 80 mg, about 100 mg, about 150 mg, about 200 mg, about 250 mg, about 500 mg, about 750 mg, or about 1000 mg per day.

Further, by way of example only, the dose of the compound for methods of treating a disease as described herein may be a fixed dose, e.g., a fixed dose of 10 mg, 15 mg, 20 mg, 25 mg, 30 mg, 35 mg, 40 mg, 45 mg, 50 mg, 55 mg, 60 mg, 65 mg, 70 mg, 75 mg, or 80 mg.

### Methods of Treatment

The methods described herein are useful for the inhibition of kinase activity of one or more enzymes. In some embodiments the kinase is TYK2.

Provided herein are methods for treating a disease or disorder, wherein the disease or disorder is an autoimmune disorders, inflammatory disorders, proliferative disorders, endocrine disorders, neurological disorders, or disorders associated with transplantation, said method comprising administering to a patient in need thereof, an effective amount of a compound or composition (e.g., an oral tablet composition) described herein.

In some embodiments, the disease or disorder is an autoimmune disorder. In some embodiments the disease or disorder is selected from type 1 diabetes, systemic lupus erythematosus, multiple sclerosis, psoriasis, Behçet's disease, POEMS syndrome, Crohn's disease, ulcerative colitis, and inflammatory bowel disease.

In some embodiments, the disease or disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disease or disorder is a proliferative disorder. In some embodiments, the proliferative disorder is cancer. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disease or disorder is an endocrine disorder. In some embodiments, the endocrine disorder is polycystic ovary syndrome, Crouzon's syndrome, or type 1 diabetes.

In some embodiments, the disease or disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disease or disorder is associated with transplantation. In some embodiments the disease or disorder associated with transplantation is transplant rejection, or graft versus host disease.

In some embodiments the disease or disorder is associated with type I interferon, IL-10, IL-12, or IL-23 signaling. In some embodiments the disease or disorder is associated with type I interferon signaling. In some embodiments the disease or disorder is associated with IL-10 signaling. In some embodiments the disorder is associated with IL-12 signaling. In some embodiments the disease or disorder is associated with IL-23 signaling.

Provided herein are methods for treating an inflammatory or allergic condition of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Provided herein are methods for treating other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease is acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), or osteoarthritis.

In some embodiments the inflammatory disease is a Th1 or Th17 mediated disease. In some embodiments the Th17 mediated disease is selected from Systemic lupus erythematosus, Multiple sclerosis, and inflammatory bowel disease (including Crohn's disease or ulcerative colitis).

In some embodiments the inflammatory disease is Sjogren's syndrome, allergic disorders, osteoarthritis, conditions of the eye such as ocular allergy, conjunctivitis, keratoconjunctivitis sicca, vernal conjunctivitis, or diseases affecting the nose such as allergic rhinitis.

### Combination Therapy

In certain instances, a compound or composition (e.g., a disclosed oral tablet composition) described herein is administered in combination with a second therapeutic agent, for example, a proton pump inhibitor, e.g., rabeprazole.

In some embodiments, the benefit experienced by a patient is increased by administering a compound described herein with a second therapeutic agent (which also includes a therapeutic regimen) that also has therapeutic benefit.

In one specific embodiment, a compound or composition described herein is co-administered with a second therapeutic agent, wherein the compound or composition described herein and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of a compound disclosed herein will be utilized in formulating a pharmaceutical composition (e.g., an oral tablet composition and/or in treatment regimens when the compounds disclosed herein are administered in combination with a second therapeutic agent. Therapeutically-effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound or composition is initiated prior to, during, or after treatment with a second agent described herein, and continues until any time during treatment with the second agent or after termination of treatment with the second agent. It also includes treatments in which a compound or composition and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g., the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds and compositions describe herein are administered before, during, or after the occurrence of a disease or condition, and the timing of administering the composition containing a compound varies. Thus, in one embodiment, the compounds and compositions described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound or composition described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound or composition described herein is administered in combination with an adjuvant. In one embodiment, the therapeutic effectiveness of one of the compounds or compositions described herein is enhanced by administration of an adjuvant (e.g., by itself the adjuvant has minimal therapeutic benefit, but in combination with another therapeutic agent, the overall therapeutic benefit to the patient is enhanced).

### EXAMPLES

The following examples are provided for illustrative purposes only, and are not intended to limit the scope of the disclosure.

N-(4-((2-Methoxy-3-(1-(methyl-d₃)-1H-1,2,4-triazol-3-yl)phenyl)amino)-5-(propanoyl-3,3,3-d₃)pyridin-2-yl)cyclopropanecarboxamide (Compound A) can be prepared according to the synthetic procedures described in WO 2020/086616, the entire content of which is incorporated by reference herein.

### Example 1

### Study Description

The relative bioavailability of Compound A as an oral tablet composition versus liquid formulation, and the effect of food or gastric acid reduction on the pharmacokinetics of a disclosed compound or oral tablet composition, in healthy participants is investigated.

### Primary Objectives

Assessing the relative bioavailability of 60 mg Compound A administered as two 30 mg immediate release tablets versus a liquid formulation in healthy participants; assessing the effect of food on the pharmacokinetics (PK) of 60 mg Compound A administered as two immediate-release 30 mg tablets in healthy participants; assessing the effect of gastric acid reduction with rabeprazole on the PK of 60 mg Compound A administered as two immediate-release 30 mg tablets in healthy participants. Secondary objectives include assessing the safety and tolerability of single 60 mg Compound A dose in healthy participants and/or assessing the effect of a single 60 mg Compound A dose on selected pharmacodynamic (PD) markers in healthy participants.

### Study Design

The study is a single-center, in-house, open-label, 4-period, partially randomized crossover study in approximately 15 healthy participants. The study population includes healthy, male and female participants, 18 to 60 years of age with a body mass index (BMI) between 18 and 34 kg/m2. The estimated study duration includes: The screening period: Up to 28 days; Treatment period: 17 days; and Follow-up period: 6 to 10 days after the last dose.

Each participant will receive a single Compound A dose on 4 occasions:
A. 60 mg liquid formulation administered in the fasted state.
B. 60 mg (two 30 mg tablets) administered in the fasted state.
C. 60 mg (two 30 mg tablets) administered with a high calorie breakfast.
D. 60 mg (two 30 mg tablets) administered in the fasted state on Day 5 of a 5-day course of rabeprazole 20 mg once daily.

Regimen D will occur last, but the sequence of Regimens A, B and C will be randomized (ABC, BCA, CAB). Eligible participants are admitted to the clinical research unit (CRU) on the day prior to the first study drug administration (Day −1, Period 1) and remain in the CRU for 18 days. An End of Study (EOS) visit will occur 6 to 10 days after the last Compound A administration.
Fasted Compound A Administrations (A and B):

Compound A 60 mg as two 30 mg tablets or 60 mg as a liquid formulation is administered with 240 mL water after an overnight fast of at least 10 hours and is followed by another 4 hour fast. Water is allowed ad libitum except for 1 hour before and 1 hour after Compound A administration.
Fed Compound A Administrations (C):

Compound A 60 mg as two 30 mg tablets are administered with 240 mL water at the end of a standardized high calorie meal. The meal is administered after an overnight fast of at least 10 hours and followed by another 4 hour fast. Water is allowed ad libitum except for 1 hour before and 1 hour after Compound A administration.
Fasted rabeprazole and fasted Compound A Administrations (D):

Rabeprazole 20 mg is administered with 240 mL water after at least a 10 hour overnight fast on Days 3, 4, 5 and 6 of Period 3; administration is followed by another 1 hour fast. On Day 1 of Period 4, rabeprazole 20 mg is administered together with Compound A 60 mg (two 30 mg tablets) with 240 mL water after an overnight fast of at least 10 hours and is followed by another 4 hour fast. Water is allowed ad libitum except for 1 hour before and 1 hour after rabeprazole and/or Compound A administration.

The following PK parameters for Compound A are determined, as appropriate:
Cₘₐₓ: Maximum plasma Compound A concentration determined directly from the concentration-time profile
Tₘₐₓ: Time of maximum plasma Compound A concentration determined directly from the concentration-time profile.
AUC₀₋ₗₐₛₜ: Area under the concentration-time curve from pre-dose (time 0) to the time of the last quantifiable concentration (tₗₐₛₜ).
AUC_{0-inf}: Area under the concentration-time curve from pre-dose (time 0) extrapolated to infinite time (AUC₀₋ₗₐₛₜ+C_{last/λz})
AUC_{% extrap}: Percentage of AUC_{0-inf} that is due to extrapolation beyond tₗₐₛₜ.
λ_{z}: The terminal elimination rate constant determined by selection of at least three data points on the terminal phase of the concentration-time curve
t_{1/2}: Terminal elimination half-life calculated as: In2/λ_{z}
CL/F: Apparent total body clearance calculated as: Dose/AUC_{inf}
Vz/F: Apparent volume of distribution calculated as: Dose/(AUC_{inf}*λ_{z})
Ae₀₋₄₈: Cumulative urinary excretion of unchanged drug in a complete collection up to 48 hours calculated as: Compound A urine concentration*urine volume
% Fe: Percentage of drug excreted unchanged in the urine calculated as: (Ae/Dose)*100
CLr: Renal clearance of the unchanged drug from plasma calcd as Ae₀₋₄₈/AUC₀₋₄₈:

Participants who meet the following criteria will be considered eligible to participate in the clinical study:

Participant is able to understand and comply with the study procedures, understand the risks involved in the study, and provide written informed consent according to federal, local, and institutional guidelines before any clinical screening activities; Participant is a male or female between the ages of 18 and 60 years, inclusive, at the screening visit; Participant has a BMI between 18 kg/m2 and 34 kg/m2, inclusive, at the screening visit; Participant is healthy as determined by medical history, physical examination, vital signs, and routine laboratory parameters (chemistry, hematology, serology, and urinalysis), and ECG at the Screening Visit and on Day −1. Laboratory values outside the normal range are acceptable if deemed to be clinically insignificant. Screening assessments may be repeated 1 time at the investigator's discretion; Female participants must not be pregnant (negative pregnancy test for women of childbearing potential at screening and check-in) or lactating and, if sexually active, must fulfill the following criteria: If of childbearing potential, must use a highly effective method (<1% failure rate) of birth control (e.g., non-hormonal intrauterine device, azoospermic partner, sexual abstinence) during participation in the study until at least 3 months after the last dose of study drug, or is surgically sterile (i.e., hysterectomy, bilateral tubal ligation or bilateral oophorectomy) at least 6 months prior to the first dose of study drug, or is postmenopausal (defined as amenorrhea 12 consecutive months and documented plasma follicle-stimulating hormone (FSH) level>40 IU/mL). Sexually active male participants with female sexual partners must be surgically sterile for 6 months (e.g., vasectomy) or use a condom together with spermicidal foam/gel/film/cream/suppository from the Screening Visit through 3 months after the last dose of study drug (even with female partners of non-childbearing potential to prevent seminal drug transmission) and must refrain from sperm donation throughout the same time period.

Participants who meet one or more of the following criteria will not be considered eligible to participate in the clinical study: Participant has a prior exposure to Compound A; Participant has a history of hypersensitivity to any of the ingredients of Compound A or rabeprazole; Participant has a history or presence of immunosuppression or significant cardiovascular, pulmonary, hepatic, renal, hematological, gastrointestinal, endocrine, immunologic, dermatologic or neurological disease, including any acute illness or major surgery within the past 3 months determined by the investigator to be clinically significant; Participant has a history of gastrointestinal, liver (including Gilbert's syndrome), kidney, or other conditions known to interfere with the absorption, distribution, metabolism, or excretion of drugs (s/p cholecystectomy is permitted); Participant has a history of malignancy within 10 years of Day −1 (5 years for in situ cervical cancer and two years for surgically excised nonmelanomatous skin cancers); Participant has a positive serologic test at Screening for infection with human immunodeficiency virus (HIV-1 and HIV-2), hepatitis B surface antigen (HBsAg) or hepatitis C virus (HCV) antibodies; Participant has a history of active, latent or inadequately treated tuberculosis infection or a positive Quantiferon test at Screening; Participant has a positive test for alcohol or drugs of abuse at Screening or Day −1; Participant has used prescription medication within 28 days of Day −1 or a vaccination within 14 days of Day −1 or OTC medication (including herbal preparations and supplements) within 14 days of Day −1 (acetaminophen up to 1.5 g per day is allowed); Participant has used or plans to use systemic immunosuppressive (e.g., corticosteroids, methotrexate, azathioprine, cyclosporine) or immunomodulating medications (e.g., IFN) during the study or within 3 months prior to the first study drug administration; Participant has a history or evidence of any other clinically significant disorder, condition, or disease that, in the opinion of the investigator, would pose a risk to participant safety or interfere with the study evaluation, procedures, or completion; Participant has any condition or treatment for a condition that might interfere with the conduct of the study or would, in the opinion of the investigator, put the participant's participation in the study at risk. This includes, but is not limited to, alcoholism, drug dependency or abuse, and psychiatric conditions; Participant is currently using any tobacco or nicotine containing products exceeding 10 cigarettes per day or equivalent; Participant received an investigational drug (or is currently using an investigational device) within 30 days prior to Screening, or at least 5 times the respective elimination half-life (t1/2), whichever is longer; Participant is unable to comply with the study restrictions/requirements, including the ingestion of the high calorie meal, e.g., has lactose intolerance, allergy to eggs, is a vegetarian; Participant has donated 500 mL or more of blood in the last 60 days or plasma in the last two weeks prior to the Screening Visit.

Dosing Day: Dosing for Regimens A, B, and D will take place after an overnight fast of at least 10 hours. Water intake will be allowed up to 1 hour before dosing and from 1 hour after dosing. Fasting restrictions continue (no food or drink other than water) for at least 4 hours post-dose. Compound A is administered with 240 ml of water at the end of the meal.

Fed conditions: A standardized high calorie breakfast is administered after an overnight fast of at least 10 hours and should be ingested approximately within 30 minutes. Compound A is administered with 240 ml of water at the end of the meal.

### Example 2: Study Description

A blinded, randomized, placebo-controlled, multiple dose study to determine the safety, tolerability, pharmacokinetics, and pharmacodynamics of Compound A tablets administered over 14 days to healthy participants is conducted.

### Primary Objectives

To assess the safety and tolerability of Compound A tablets administered over 14 days to healthy participants; to assess the pharmacokinetics (PK) of Compound A tablets in healthy participants at steady-state.

### Study Design

This is a single-center, in-house, double-blind with sponsor unblinded, randomized, placebo-controlled, parallel-group, multiple dose study in up to 6 cohorts of healthy participants. Participants will be admitted to the clinical research unit on the day prior to the first dose (Day −1) of investigational medicinal product (IMP) and remain until 48 hours after the last dose of a 14-day course of IMP. In case of more than once daily administration, the last dose will be the first (morning) dose on Day 14. An End of Study visit will occur 5 to 7 days after the last dose of IMP. The number of participants will be approximately 48 healthy adult participants (up to 6 cohorts of 8 participants each, in a randomization ratio of 3:1 to Compound A versus placebo). Healthy male and female participants, 18 to 60 years of age with a body mass index (BMI) between 18 and 32 kg/m2, are planned for enrollment.

IMPs will consist of Compound A tablets of 10 and 30 mg and their matching placebo. Single doses of up to 100 mg Compound A have been administered to healthy participants in the FIH study without clinically significant safety concerns. Cₘₐₓ at that dose averaged 949 ng/ml (highest value 1,160 n/mL) and AUC0-inf averaged 11,286 ng*h/mL (highest value 13,705 ng*h/ml). Seven-day courses of up to 40 mg q12h (mean Cₘₐₓ at steady state [C_{max,ss}]433 ng/mL, mean AUC from time 0 to 12 hours at steady state [AUCo-12,ss] 3620 ng*h/mL) have also been studied and were well tolerated in that study. Dose levels for the first 2 cohorts will be 20 mg qd and 20 mg q12h, respectively. Other dose levels will depend on the relative bioavailability of the tablet vs. the suspension. Administration may occur qd or q12h and the maximum dose to be studied will have a predicted mean steady state Cₘₐₓ and AUC₀₋₂₄ that does not exceed 949 ng/ml and 11,286 ng*h/mL, respectively. Assuming bioequivalence between the tablet and the suspension, the maximum dose to be studied would be 60 mg q12h or 80 mg qd. If there is a 50% reduction in bioavailability of the tablet vs. the suspension, the maximum dose to be studied would be 120 mg q12h or 160 mg qd; these are the highest potential doses planned for this study.

The following safety variables will be recorded at regular intervals during the study: Adverse events (AEs); Clinical laboratory tests (hematology, clinical chemistry, and urinalysis); Vital signs (systolic and diastolic blood pressure, body temperature, pulse, and respiratory rate); 12-lead electrocardiogram (ECG) and continuous Holter ECGs; and Physical examinations. Pharmacokinetic endpoints to be recorded will be: Maximum observed concentration (Cₘₐₓ) after the first and the last dose; Time to Cₘₐₓ (Tₘₐₓ) after the first and the last dose; AM trough concentrations (C_{trough}) on Days 3, 5, 7, 9, 12, 13, and 14; Area under the concentration-time curve from 0 to time of last quantifiable concentration (AUC₀₋ₜ) after the last dose; Area under the concentration-time curve during the dosing interval after the first and the last dose (AUC₀₋ₜₐᵤ); Accumulation ratio (RAUC=AUC₀₋ₜₐᵤ after the last dose/AUC₀₋ₜₐᵤ after the first dose); Accumulation ratio calculated using Cₘₐₓ (Rac[Cₘₐₓ]); Apparent terminal elimination half-life (t_{1/2}) after the last dose; Terminal elimination rate constant (λ_{z}) after the last dose; Total apparent body clearance (CL/F) following oral administration at steady state; Apparent steady state volume of distribution (Vₛₛ/F); Ae₀₋₂₄: Cumulative urinary excretion of unchanged drug in a complete 24-hour collection calculated as: Compound A urine concentration*urine volume; % Fe: Percentage of drug excreted unchanged in the urine calculated as: (Ae₀₋₂₄/Dose administered over 24 hours)*100; CLr: Renal clearance of the unchanged drug from plasma calculated as: Ae₀₋₂₄/AUC₀₋₂₄ for qd cohorts and Ae₀₋₂₄/2*AUC₀₋₁₂ for q12h cohorts.

### Example 3:

### Study Description

A randomized, double-blind, placebo-controlled study to evaluate the efficacy and safety of Compound A in patients with moderate to severe plaque psoriasis is conducted.

### Primary Objectives

To compare the Psoriasis Area and Severity Index (PASI-75) between doses of Compound A and placebo after 12 weeks of treatment.

### Primary Endpoints

Proportion of patients with moderate to severe psoriasis achieving≥75% reduction in PASI score at 12 weeks between doses of Compound A and placebo.

### Secondary Objectives

To assess the safety and tolerability of Compound A dose in moderate to severe psoriasis patients.; To characterize the pharmacokinetics (PK) of Compound A; To assess the response rate in static Physician's Global Assessment (sPGA) score after 12 weeks of treatment; To assess the response rate in PASI-50, 90, and 100 score after 12 weeks of treatment; To compare the response rate in PASI-75 among Compound A treatments; To assess the effect on body surface area (% BSA) involved with psoriasis after 12 weeks of treatment; To assess the change in Dermatology Life Quality Index (DLQI).

### Secondary Endpoints

Incidence of treatment-emergent adverse events (TEAEs) and serious adverse events (SAEs); Plasma concentrations and PK parameters of Compound A; Proportion of patients achieving an sPGA score of “0” (“cleared”) or “1” (“minimal”) after 12 weeks of treatment compared with placebo; Proportion of patients achieving PASI-50, 90, and 100 after 12 weeks of Compound A treatment compared with placebo; Proportion of patients achieving PASI-75 at 12 weeks compared among the Compound A treatments; Change from baseline in % BSA after 12 weeks of Compound A treatment compared with placebo; Change from baseline in DLQI at week 12 in Compound A compared with placebo.

### Exploratory Objectives

To assess the change in pruritis numerical rating scale (NRS) after 12 weeks of treatment; To assess the change in EQ-5D; To assess the change in psoriasis and TYK2-related skin-based biomarkers with Compound A treatment; To assess the change in psoriasis and TYK2-related blood-based biomarkers with Compound A treatment; To assess the change transcriptomic and proteomic-based biomarkers in blood and skin with Compound A treatment.

### Exploratory Endpoints

Change from baseline in NRS score after 12 weeks of Compound A treatment compared with placebo; Change from baseline in EQ-5D at week 12 in Compound A compared with placebo; Change from baseline in psoriasis and TYK2-related skin biomarkers in response to Compound A treatment; Change from baseline in psoriasis and TYK2-related skin biomarkers in response to Compound A treatment; Change from baseline in transcriptomic and proteomic expression in response to Compound A treatment.

### Study Design

This is a randomized, double-blind, placebo-controlled study in patients with moderate to severe psoriasis. The purpose of this study is to assess the clinical efficacy, safety, PK, and PD of Compound A compared to placebo in patients with moderate to severe psoriasis. The overall study duration will be approximately 20 weeks. The screening period will be 4 weeks, the treatment period will be 12 weeks, and the safety follow-up period will be 4 weeks. For the entire study, study completion is defined as the last visit of the last patient for any protocol related activity (last patient, last visit). For individual patients, study completion is defined as the time of the patient's last data collection. Efficacy (including PASI, sPGA, QOL measures), safety, and PK will be assessed. Exploratory pharmacodynamic (PD) assessments include blood-and skin-based RNA, proteomic, and other molecular markers. Continued treatment will be offered in a separate open-label extension study.

The study population will consist of male and female adult patients with moderate to severe psoriasis. Patients must meet all the inclusion criteria and none of the exclusion criteria. Patients fulfilling all eligibility criteria will be randomized in a 1:1:1:1:1:1 manner to receive oral doses of Compound A at one of 5 dose levels (10 mg QD, 20 mg QD, 40 mg QD, 20 mg BID, 40 mg BID) or placebo. Approximately 210 patients will be randomly assigned to one of the 6 study arms (35 patients per arm). Compound A is provided as tablets in two dose strengths, 10 mg and 20 mg. Matching placebo tablets are similar in size, shape, and appearance to the Compound A 10 mg and 20 mg active drug tablets. Compound A dose levels administered in each arm of the study are outlined in Table 1. Patients will take assigned blinded Compound A or placebo.

TABLE 1

| | | | | | Number of Tablets Per Dose | | | | | |
|---|---|---|---|---|---|---|---|---|---|---|
| | | | Dose | Dose | AM | | | PM | | |
| Arm | Drug | Route | Level | Regimen | 10 mg | 20 mg | PBO | 10 mg | 20 mg | PBO |
| 1 | Cmpd A | Oral | 10 mg | QD | 1 | — | 1 | — | — | 2 |
| 2 | Cmpd A | Oral | 20 mg | QD | — | 1 | 1 | — | — | 2 |
| 3 | Cmpd A | Oral | 40 mg | QD | — | 2 | — | — | — | 2 |
| 4 | Cmpd A | Oral | 20 mg | BID | — | 1 | 1 | — | 1 | 1 |
| 5 | Cmpd A | Oral | 40 mg | BID | — | 2 | — | — | 2 | — |
| 6 | Placebo | Oral | — | BID | — | — | 2 | — | — | 2 |

Inclusion criteria includes those patients with a diagnosis of plaque psoriasis for ≥6 months; Plaques covering≥10% of body surface area (BSA); Psoriasis area severity index score (PASI) score≥12 and static Physician's Global Assessment (sPGA) score≥3.

Diagnosis of non-plaque psoriasis (guttate, inverse, pustular, erythrodermic, drug induced) and/or diagnosis of or other immune-mediated conditions that are commonly associated with psoriasis (e.g., uveitis, inflammatory bowel disease) or other inflammatory skin conditions that may interfere with the study assessment; Diagnosis of psoriatic arthritis, currently requiring systemic (oral, SC, IM or IV) immunosuppressant medical treatment (including corticosteroids, immunosuppressants, biologics) or who has or is expected, in the opinion of the investigator, to develop unstable disease, may be criteria for non-inclusion in the study.

### Example 4

### Study Description

A phase 2, randomized, double-blind, placebo-controlled study to assess the safety, efficacy, and pharmacokinetics of multiple dose levels of compound A in adult patients with systemic lupus erythematosus (SLE) is conducted.

### Primary Objectives

To compare the effect on disease activity measured by the proportion of patients achieving British Isles Lupus Assessment Group (BILAG)-based Composite Lupus Assessment (BICLA) response at week 32 between doses of Compound A and placebo

### Primary Endpoints

Composite endpoint (BICLA), defined by meeting all of the following criteria: Reduction of all BILAG-2004 A to B/C/D and baseline BILAG-2004 B to C/D, and no BILAG-2004 worsening in other organ systems, as defined by ≥1 new BILAG-2004 A or ≥2 new BILAG-2004 B; No worsening from baseline in SLE Disease Activity Index 2000 (SLEDAI-2K), where worsening is defined as an increase from baseline of >0 points in SLEDAI-2K; No worsening from baseline in physician's assessment of patient lupus disease activity, where worsening is defined by an increase≥0.30 points on a 3-point Physician's Global Assessment (PGA) visual analogue scale (VAS); No discontinuation of investigational product (IP) or use of rescue medication beyond the protocol-allowed threshold before assessment; The estimand is the difference in proportions of treatment successes between treatment conditions (doses of Compound A and placebo, regardless of discontinuation) in patients with moderate to severe disease SLE whereby treatment success is defined as response at week 32 in BICLA without use of additional or alternative medications.

### Secondary Objectives

To assess the safety and tolerability of multiple dose levels of Compound A; To compare the effect on cutaneous disease activity measured by the proportion of patients with a Cutaneous Lupus Erythematosus Disease Area and Severity Index (CLASI) activity score of ≥8 at baseline achieving≥50% reduction in the CLASI activity score at week 32 between doses of Compound A and placebo; To compare the effect on disease activity measured by the proportion of patients achieving BICLA response at week 32 between doses of Compound A and placebo in the interferon gene signature (IGS) high subgroup; To compare the effect on disease activity measured by the proportion of patients achieving an SLE Responder Index of ≥4 (SRI[4]) response at week 32; To compare the Lupus Low Disease Activity State (LLDAS) response between doses of Compound A and placebo at week 32; To compare the steroid use in patients at week 32; To compare the effect on health-related quality of life (HRQOL) between doses of Compound A and placebo; To compare disease-specific QoL between doses of Compound A and placebo; To compare fatigue measured by FACIT-F between doses of Compound A and placebo; To compare patient global assessment of disease activity (PtGA) between doses of Compound A and placebo.

### Secondary Endpoints

Incidence of treatment-emergent AEs (TEAEs) and SAEs, vital signs, physical examination, 12-lead ECG, and clinical laboratory tests (hematology, clinical chemistry, urinalysis); 50% reduction in CLASI activity score compared to baseline defined by the following criteria: Baseline CLASI activity score≥8 and ≥50% reduction of CLASI activity score at week 32 compared to baseline; No discontinuation of IP or use of rescue medication beyond the protocol-allowed threshold before assessment; BICLA response; Composite endpoint SRI(4), defined by the following criteria: Reduction from baseline of ≥4 points in the SLEDAI-2K, and no new BILAG A or more than 1 BILAG B domain score, and no deterioration from baseline≥0.3 in the PGA; No discontinuation of IP or use of rescue medication beyond the protocol-allowed threshold before assessment; LLDAS measured by achieving the following: SLEDAI-2K≤4, with no activity in major organ systems and no hemolytic anemia or gastrointestinal activity, and No new lupus disease activity compared with the previous SLEDAI-2K assessment, and PGA≤1, and Current prednisone (or equivalent) dose≤7.5 mg daily; Well-tolerated standard maintenance doses of immunosuppressive drugs and approved biological agents; Proportion of patients who achieve steroid dose of ≤5 mg/day prednisone equivalent; Difference between baseline (Day1) and week 32 in the Short Form 36 Health Survey, version 2 acute (SF-36 v.2 acute) physical (PCS) and/or mental (MCS) component summary scores; Difference between baseline (Day1) and week 32 in the LupusQoL; Difference between baseline (Day1) and week 32 in FACIT-F; Difference between baseline (Day1) and week 32 in PtGA.

### Week 48 Secondary Analyses Objectives

To compare BICLA response between doses of Compound A and placebo at week 48; To compare the effect on cutaneous disease activity measured by the proportion of patients with a CLASI activity score of ≥8 at baseline achieving≥50% reduction in the CLASI activity score at week 48 between doses of Compound A and placebo; To compare the effect on disease activity measured by the proportion of patients achieving BICLA responses at week 48 between doses of Compound A and placebo in the IGS high subgroup; To compare the SRI(4) response between doses of Compound A and placebo after 48 weeks of treatment; To compare the LLDAS response between doses of Compound A and placebo at weeks 48; To compare the annualized flare rate through Week 48; To compare glucocorticoid use in patients at week 48; To compare the effect on HRQOL between doses of Compoun A and placebo; To compare disease specific QoL between doses of Compound A and placebo; To compare Fatigue measured by FACIT-F between doses of Compound A and placebo; To compare patient global assessment of disease activity (PtGA) between doses of Compound A and placebo.

### Week 48 Secondary Analyses Endpoints

BICLA response at week 48; CLASI at week 48; SRI(4) at week 48; LLDAS at week 48; Annualized flare rate with flare defined as ≥1 BILAG A or ≥2 BILAG B scores due to items rated as new or worse; Proportion of patients who achieve glucocorticoid doses of ≤5 mg/day prednisone equivalent; Difference between baseline (day1) and week 48 in the SF-36 v.2 acute PCS and/or MCS scores; Difference between baseline (day1) and week 48 in the LupusQoL; Difference between baseline (day1) and week 48 in FACIT-F; Difference between baseline (day1) and week 48 in PtGA.

### Exploratory Objectives

Time to Sustained BICLA response; To compare the proportion of patients with SRI(4) at weeks 32 and 48 in the IFN test-high subgroup; To characterize the PK of multiple dose levels of Compound A; To assess the change from baseline in the 28-joint count difference between Compound A and placebo; To assess the difference between Compound A and placebo on measures of disease activity including levels of SRI response, the individual components of SRI, and the number of swollen and tender joints over time up to Weeks 32 and 48, as well as SRI and BICLA over time; To assess the change from baseline in mean and median interferon regulated gene (IRG) expression levels between doses of Compound A and placebo up to 48 weeks; To assess the changes in transcriptomic-and proteomic-based biomarkers in blood with Compound A treatment; To assess the changes from baseline in mean complement (C3, C4) and anti-double stranded DNA (dsDNA) antibodies up to 48 weeks.

### Exploratory Endpoints

Defined as the visit of first BICLA response which is sustained up to and including week 48; SRI(4) response; Plasma PK parameters of Compound A up to weeks 32 and 48; Proportion of patients with a swollen or tender joint count of at least 6 at baseline who achieve 50% reduction at weeks 32 and 48; SRI(4), SRI(5), SRI(6), SRI(7), SRI(8), BICLA response over time, BILAG-2004 score, SLEDAI-2K, PGA, and joint count; Change in mean and median IRG expression levels up to 48 weeks; Change from baseline in transcriptomic and proteomic expression in response to Compound A treatment; Proportion of patients with abnormal C3 or C4 or anti-dsDNA at baseline who have normal C3 or C4 or anti-dsDNA antibodies at week 32 or week 48; Assess the effect of Compound A on measures of global SLE clinical response in patients based on IRG status (ie, high vs low IRG signature) up to 48 weeks.

### Study Design

This is a Phase 2, randomized, double-blind, placebo-controlled study to evaluate the safety, efficacy, PK, and pharmacodynamics (PD) of 3 dose levels of Compound A (20 mg once daily [QD], 40 mg [taken as 20 mg twice daily (BID)], and 60 mg [taken as 30 mg twice daily (BID)] in patients with moderately to severely active, autoantibody-positive SLE while receiving standard of care (SOC) treatment in adult patients aged 18 to 70 years of age. Randomization will be stratified using the SLEDAI-2K score at screening (<10 points vs ≥10 points), steroid dose on Day 1 (<10 mg prednisone equivalent vs ≥10 mg) and IGS score (high vs low).

The study includes a protocol-defined steroid taper. Steroid tapering will be attempted for all patients. The target is a steroid dose of ≤5 mg/day prednisone equivalent by week 24. Tapering will commence by week 8 (or earlier) and proceed until the target is reached. Tapering is not permitted between week 24 and week 32 but may resume at the Investigator's discretion after the week 32 visit up to week 40. No tapering is permitted between the week 40 and week 48 visit.

Efficacy assessments include SLEDAI-2K, BILAG-2004, PGA VAS, CLASI, tender and swollen joint counts. Patient-reported outcome (PRO) measures include SF-36 v.2 acute, Lupus QOL, Functional Assessment of Chronic Illness Therapy (FACIT)-Fatigue questionnaire and Patient Global Assessment (PtGA). Safety assessments include AE monitoring, clinical laboratory tests, vital signs, ECGs, and physical examinations. Observed concentration-time data will mainly be used for modeling and simulation purpose. PK parameters include average maximum observed concentration (Cₘₐₓ) and time to Cₘₐₓ (Tₘₐₓ); area trough observed plasma concentrations (C_{trough}) of Compound A may be determined as data permits. Exploratory PD and biomarker assessments include blood based transcriptomic, proteomic, and other TYK2-relevant and disease relevant markers.

Patients fulfilling all eligibility criteria will be randomly assigned in a 1:1:1:1 ratio to receive oral doses of Compound A at 1 of 3 dose levels or placebo, as outlined in Table 2.

TABLE 2

| Arm | Compound A Dose | Number of Patients |
|---|---|---|
| 1 | 20 mg QD | 97 |
| 2 | 40 mg (taken as 20 mg BID) | 97 |
| 3 | 60 mg (taken as 30 mg BID) | 97 |
| 4 | — | 97 |
| (Placebo) | | |

The total duration of study participation for each patient will be approximately 57 weeks. The study includes a screening period of approximately 35 days followed by a 48-week double-blind treatment period that is followed by either a 4-week safety follow-up period or an open-label extension (OLE) study for eligible patients electing to receive continued long-term treatment in the OLE study. Approximately 388 patients will be randomly assigned to 1 of the 4 study arms (3 Compound A active+1 placebo).

Participants who meet the following criteria will be considered eligible to participate in the clinical study: Able and willing to provide written informed consent (signed and dated) to participate in this study and comply with all requirements in the study protocol; Males or females, age 18 to 70 years, inclusive, at the time of informed consent; Body mass index 18 to 40 kg/m² and total body weight>40 kg (88 lbs); Adequate peripheral venous access; Diagnosed with SLE≥6 months prior to screening and fulfills the 2019 European League Against Rheumatism (EULAR)/American College of Rheumatology (ACR) criteria at the time of screening AND has either positive antinuclear antibody test at screening by immunofluorescent assay at the central laboratory with titer≥1:80 or elevated anti-dsDNA antibodies OR anti-Smith antibody at screening as determined by the central laboratory OR C3 or C4 below the lower limit of normal; During screening and prior to first administration of study drug, an SLEDAI score of >6 at screening and clinical” SLEDAI-2K score of ≥4 points (the “Clinical” SLEDAI-2K is the SLEDAI-2K assessment score without the inclusion of points attributable to any urine or laboratory results including immunologic measures (Includes points from the following clinical components: arthritis, myositis, rash, alopecia, mucosal ulcers, pleurisy, pericarditis, and vasculitis, excludes points attributable to fever, an SLE headache, and organic brain syndrome)); At least 1 of the following: BILAG-2004 Index level A disease in at least 1 body/organ system and/or BILAG-2004 Index level B disease in more than 1 body/organ system (excludes scores from Lupus Headache); PGA score of >1.0 on a 0 to 3 VAS at screening; Clinical SLEDAI-2K score≥4 points with skin involvement prior to first administration of study drug; Currently receives at least 1 of the following: dose of oral corticosteroid (≤40 mg/day prednisone or equivalent) for a minimum of 2 weeks prior to signing of the informed consent form (ICF) and/or any of the following medications administered for a minimum of 12 weeks prior to signing the ICF and at a stable dose for a minimum of 8 weeks prior to signing the ICF and until Day 1: Azathioprine≤200 mg/day, Antimalarial (eg, chloroquine, hydroxychloroquine, quinacrine), Mycophenolate mofetil≤2 g/day or mycophenolic acid≤1.44 g/day, Oral, subcutaneous, or intramuscular (IM) methotrexate≤20 mg/week (No more than 1 immunosuppressive treatment may be used concurrently and if corticosteroid is the only medication used for SLE activity, the dose must have been ≥10 mg and ≤40 mg prednisone equivalent for a minimum of 8 weeks prior to screening, and stable≥10 mg and ≤40 mg for at least 2 weeks prior to screening); Negative serum β-human chorionic gonadotropin (β-hCG) test at screening (women of childbearing potential [WOCBP] only); Screening “Clinical” SLEDAI-2K score of ≥4 points and OC dose stable for at least 2 weeks at day one prior to randomization.

Participants who meet the following general exclusion criteria will be considered ineligible to participate in the clinical study: Any acute or chronic illness/condition or evidence of an unstable clinical condition that, in the Investigator's judgment, will substantially increase the risk to the patient if he or she participates in the study; Presence or history (within 1 year) of psychiatric disease that, in the Investigator's opinion, is likely to interfere with patient's ability to provide informed consent, comply with study procedures, or place the patient at increased risk by participating in the study; Current or history within 1 year of screening of alcohol or drug abuse (excluding cannabis) based on investigator's clinical judgment; Pregnant, lactating, or planning to get pregnant during the study period and for 3 months after study completion or discontinuation; Patients with QTcF>450 msec (males) or >470 msec (females) at screening; Unstable cardiovascular disease, defined as a recent clinical deterioration (eg, unstable angina, rapid atrial fibrillation) or a cardiac hospitalization within the last 3 months; At screening, has any of the following laboratory abnormalities: absolute neutrophil count<1×10^{9/}L, hemoglobin<8 g/dL, platelet count<25,000/uL, aspartate aminotransferase (AST)/alanine aminotransferase (ALT)≥2×upper limit of normal (ULN), unless attributable to lupus hepatitis, total bilirubin≥2×ULN (unless the patient has documented Gilbert syndrome), estimated glomerular filtration rate (eGFR by Modification of Diet in Renal Disease [MDRD] formula)<30 indicative of moderate or severe renal insufficiency, and/or UPCR>3.5 g/day.

Participants who meet the following exclusion criteria related to SLE and other diseases will be considered ineligible to participate in the clinical study: Drug-induced SLE or other autoimmune diseases that, in the opinion of the Investigator, are likely to confound efficacy assessments; Active, proliferative lupus nephritis that in the investigator's opinion may require treatment not allowed by the protocol; Current disease other than SLE that, in the opinion of the Investigator, is likely to interfere with SLE disease activity assessments (examples include severe fibromyalgia, severe osteoarthritis, other severe cardiorespiratory diseases); Active severe or unstable neuropsychiatric SLE including, but not limited to the following: aseptic meningitis; cerebral vasculitis; myelopathy; demyelination syndromes (ascending, transverse, acute inflammatory demyelinating polyradiculopathy); acute confusional state; impaired level of consciousness; psychosis; acute stroke or stroke syndrome; cranial neuropathy; new seizures; cerebellar ataxia; and mononeuritis multiplex that would make the patient unable to fully understand the ICF, or where in the opinion of the Principal Investigator protocol-specified SOC is insufficient and utilization of a more aggressive therapeutic approach (e.g., adding intravenous (IV) cyclophosphamide and/or high dose IV pulse corticosteroid therapy or other treatments not permitted in the protocol) is indicated; History of or current diagnosis of catastrophic antiphospholipid syndrome within 12 months prior to signing the ICF (antiphospholipid syndrome adequately controlled by anticoagulant therapy for at least 3 months is acceptable and positive antiphospholipid antibodies without clinical symptoms are not exclusionary); History of any recurrent non-SLE disease that required treatment with oral or parenteral corticosteroids for more than a total of 2 weeks within the last 52 weeks prior to signing the ICF (treatment with steroids for conditions that are not expected to recur (eg, poison ivy) is not excluded).

Participants who meet the following exclusion criteria related to concomitant medications will be considered ineligible to participate in the clinical study: Receipt of any investigational products (small molecule or biologic agent) within 3 months or 5 half-lives, whichever is greater, prior to signing of the ICF, or is currently enrolled in an investigational study; Receipt of any commercially available biologic agent within 5 half-lives prior to signing of the ICF; Receipt of B cell-depleting therapy (including but not limited to obinutuzumab, ocrelizumab, or rituximab)<6 months prior to signing the ICF or, if therapy was administered>6 months prior to signing the ICF, absolute B cell less than the lower limit of normal or baseline value prior to receipt of B cell-depleting therapy (whichever is lower); Has received any therapeutic agent targeted to IL-12, IL-17, or IL-23 within 6 months or any tumor necrosis factor alpha inhibitor(s) within 12 weeks of prior to signing the ICF; Has received JAK inhibitors (eg, baricitinib, tofacitinib) or TYK2 inhibitors within 12 weeks of prior to signing the ICF; Is currently receiving PPIs such as omeprazole or esomeprazole (it is acceptable to substitute a histamine 2 receptor blocking drug, or oral antacids prior to study day 1); Receipt of any of the following: Intra-articular, IM, or IV glucocorticosteroids within 6 weeks prior to Day 1, any live or attenuated vaccine within 4 weeks prior to signing the ICF (administration of killed vaccines is acceptable), blood transfusion within 4 weeks prior to signing the ICF, and/or any of the following restricted medications if the washout period is not met: abatacept (12 wks), adalimumab (12 wks), anakinra (4 wks), anifrolumab (12 wks), belimumab (12 wks), BTK inhibitors (12 wks), certolizumab pegol (12 wks), cyclophosphamide (IV or oral, 24 wks), cyclosporine (oral, 8 wks), danazol (4 wks), dapsone (4 wks), eculizumab (8 wks), etanercept (12 wks), golimumab (12 wks), infliximab (12 wks), IV immunoglobulin (8 wks), JAK inhibitors (12 wks), leflunomide (36 wks or 2 wks after cholestyramine washout), lenalidomide (8 wks), natalizumab (12 wks), ocrelizumab (48 wks), pimecrolimus (4 wks), plasmapheresis (8 wks), retinoids (4 wks), rituximab (48 wks), ruxolitinib (4 wks), B-cell depleter (48 wks), sulfasalazine (4 wks), sirolimus (4 wks), tacrolimus (12 wks), thalidomide (8 wks), tocilizumab (12 wks), investigational agents (30 days of 5 half lives).

Participants who meet the following exclusion criteria related to infection and other factors will be considered ineligible to participate in the clinical study: History or evidence of suicidal ideation (severity of 4 [active: method and intent, but no plan] or 5 [active: method, intent, and plan]) within the past 6 months; or any suicidal behavior within the past 12 months based on an assessment with the Columbia-Suicide Severity Rating Scale (C-SSRS) at screening; Known history of a primary immunodeficiency or an underlying condition such as HIV infection or splenectomy that predisposes the patient to infection; Confirmed positive test for hepatitis B serology for the following: Hepatitis B surface antigen, OR Hepatitis B core antibody (HBcAb) AND hepatitis B virus (HBV) DNA (>169 copies/mL [29 IU/mL]) detected by reflex testing at screening; Positive test for hepatitis C antibody; Any severe herpes infection at any time prior to week 0 (day 1), including, but not limited to, disseminated herpes (ever), herpes encephalitis (ever), recurrent herpes zoster (defined as 2 episodes within 2 years), or ophthalmic herpes (ever); Active herpes zoster infection within 12 weeks of prior to signing the ICF; Active herpes simplex virus within 4 weeks of day 1; Known current malignancy or current evaluation for a potential malignancy or history of malignancy within the past 5 years prior to screening, except squamous or basal cell carcinoma of the skin treated with documented success of curative therapy>3 months prior to week 0 (day 1) and/or cervical cancer in situ treated with apparent success with curative therapy>1 year prior to week 0 (day 1); Any of the following: currently active, clinically significant infection of any kind, clinically significant chronic infection (eg, osteomyelitis, bronchiectasis) within 8 weeks prior to signing the ICF (chronic nail infections are allowed), any infection requiring hospitalization or treatment with IV anti-infectives not completed at least 4 weeks prior to signing the ICF, and/or any infection requiring oral anti-infectives (including antivirals) within 2 weeks prior to day 1.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating a TYK2-mediated disease or disorder in a patient in need thereof, comprising orally administering to the patient, once or twice daily, 30 mg, 40 mg, 60 mg, or 80 mg of a compound represented by:

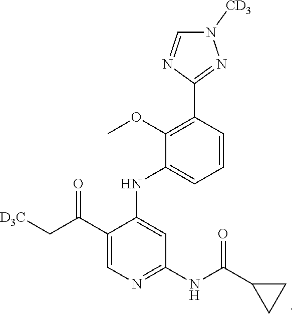 [SMILES: [2H]C([2H])([2H])CC(=O)c1cnc(NC(=O)C2CC2)cc1Nc1cccc(-c2ncn(C([2H])([2H])[2H])n2)c1OC]

2. The method of claim 1, further comprising orally administering to the patient a proton pump inhibitor.

3. The method of claim 1 or 2, wherein the proton pump inhibitor is selected from the group consisting of rabeprazole, omeprazole, esomeprazole, lansoprazole, pantoprazole, and dexlansoprazole.

4. The method of any one of claims 1-3, wherein the proton pump inhibitor is rabeprazole.

5. The method of any one of claims 1-4, wherein the compound is administered to the patient in a fasted state.

6. The method of any one of claims 1-4, further comprising administering food to the patient before administration or upon administration of the compound.

7. The method of any one of claims 1-6, wherein the TYK2-mediated disease or disorder is selected from the group consisting of Crohn's disease, rheumatoid arthritis, psoriasis, systemic lupus erythematosus, ulcerative colitis, psoriatic arthritis, and systemic sclerosis.

8. A method of treating one or more of: Crohn's disease, rheumatoid arthritis, psoriasis, systemic lupus erythematosus, ulcerative colitis, psoriatic arthritis, and systemic sclerosis in a patient in need thereof, comprising:
administering to the patient, once daily, two oral tablet compositions each comprising:
30 mg of a compound represented by: 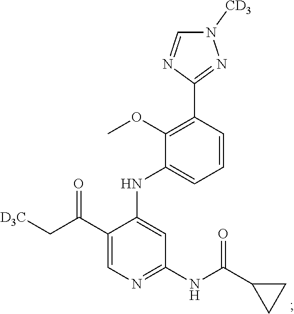 [SMILES: [2H]C([2H])([2H])CC(=O)c1cnc(NC(=O)C2CC2)cc1Nc1cccc(-c2ncn(C([2H])([2H])[2H])n2)c1OC]
and
a pharmaceutically acceptable excipient;
optionally orally administering to the patient rabeprazole; and
optionally administering food to the patient upon administration of the compound.

9. A method of treating one or more of: Crohn's disease, rheumatoid arthritis, psoriasis, systemic lupus erythematosus, ulcerative colitis, psoriatic arthritis, and systemic sclerosis in a patient in need thereof, comprising:
orally administering to the patient, once daily or twice daily, 10 mg, 20 mg, 30 mg, 60 mg, or 80 mg of a compound represented by: 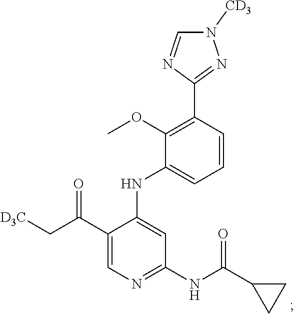 [SMILES: [2H]C([2H])([2H])CC(=O)c1cnc(NC(=O)C2CC2)cc1Nc1cccc(-c2ncn(C([2H])([2H])[2H])n2)c1OC]
wherein the patient is in a fasted state or a fed state.

10. A fixed unit dose pharmaceutical composition for oral administration comprising:
10 mg, 20 mg, 30 mg, 40 mg, 60 mg, or 80 mg of a compound represented by: 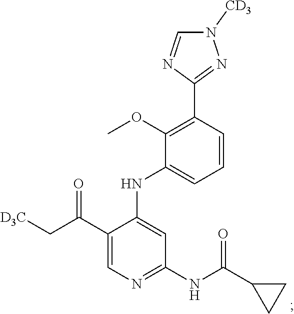 [SMILES: [2H]C([2H])([2H])CC(=O)c1cnc(NC(=O)C2CC2)cc1Nc1cccc(-c2ncn(C([2H])([2H])[2H])n2)c1OC]
and
a pharmaceutically acceptable excipient.

11. The pharmaceutical composition of claim 10, wherein the composition is formulated as a tablet.

12. An oral tablet composition comprising:
about 30 mg of a compound represented by: 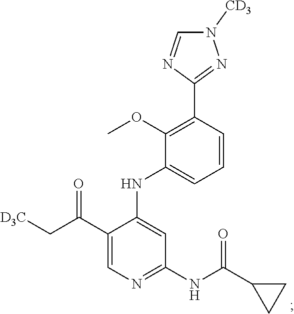 [SMILES: [2H]C([2H])([2H])CC(=O)c1cnc(NC(=O)C2CC2)cc1Nc1cccc(-c2ncn(C([2H])([2H])[2H])n2)c1OC]
and
a pharmaceutically acceptable excipient.

13. A method of treating a TYK2-mediated disease or disorder in a patient in need thereof, comprising administering to the patient an effective amount of the oral tablet composition of claim 12.

14. The method of claim 13, wherein the administrating is once daily administering.

15. A method of treating a TYK2-mediated disease or disorder in a patient in need thereof, comprising orally administering to the patient, once or twice daily, 10 mg, 20 mg, 30 mg, 40 mg, 80 mg, 100 mg, 120 mg, or 160 mg of a compound represented by:

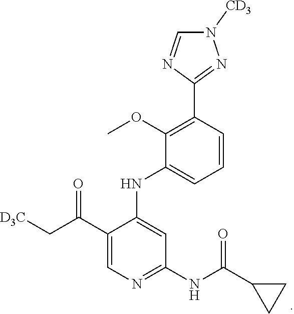 [SMILES: [2H]C([2H])([2H])CC(=O)c1cnc(NC(=O)C2CC2)cc1Nc1cccc(-c2ncn(C([2H])([2H])[2H])n2)c1OC]

16. A method of treating moderate to severe psoriasis in a patient in need thereof, comprising orally administering to the patient, once or twice daily, 10 mg, 20 mg, 30 mg, 40 mg, 80 mg, or more of a compound represented by: 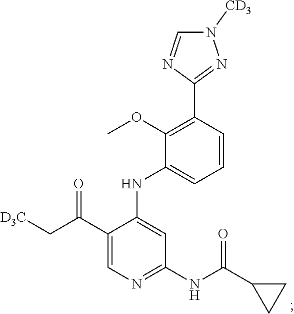 [SMILES: [2H]C([2H])([2H])CC(=O)c1cnc(NC(=O)C2CC2)cc1Nc1cccc(-c2ncn(C([2H])([2H])[2H])n2)c1OC]
wherein after at least 12 weeks of administration the patient has significant clinical improvement as characterized by the Area and Severity Index (PASI-75) score as compared to baseline.

17. A method of treating moderate to severe psoriasis in a patient in need thereof, comprising orally administering to the patient, once or twice daily, 10 mg, 20 mg, 30 mg, 40 mg, 80 mg, or more of a compound represented by: 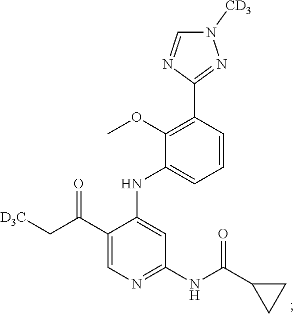 [SMILES: [2H]C([2H])([2H])CC(=O)c1cnc(NC(=O)C2CC2)cc1Nc1cccc(-c2ncn(C([2H])([2H])[2H])n2)c1OC]
wherein after at least 12 weeks of administration the patient has significant clinical improvement as characterized by % effect on body surface area involved with psoriasis.

18. The method of claim 16 or 17, wherein the patient, before administration, has at least one of: a diagnosis of plaque psoriasis for ≥6 months; plaques covering≥10% of body surface area (BSA); psoriasis area severity index score (PASI) score≥12 and/or a static Physician's Global Assessment (sPGA) score of ≥3.

19. The method of any one of claims 16-18, wherein 10 mg, 20 mg, 40 mg of the compound is administered once or twice daily.
